(19) United States
(12) Patent Application Publication
(10) Pub. No.: US 2011/0111013 A1
(43) Pub. Date: May 12, 2011

(54) TRANSDERMAL THERAPEUTIC SYSTEM FOR ADMINISTRATION OF FENTANYL OR AN ANALOGUE THEREOF

(75) Inventors: Nouha Salman, Muenchen (DE); Ingo Teutsch, Muenchen (DE)

(73) Assignee: ACINO AG, Miesbach (DE)

(21) Appl. No.: 12/988,174

(22) PCT Filed: Apr. 24, 2009
(86) PCT No.: PCT/EP2009/003010

§ 371 Date: Jan. 5, 2011

(30) Foreign Application Priority Data
Apr. 25, 2008 (EP) 08155167.3

(51) Int. Cl. A61K 9/70; A61K 31/4468; A61K 31/4535; A61P 25/04; A61P 29/00

(52) U.S. Cl. 424/449; 514/329; 514/326

(57) ABSTRACT

What is disclosed is a transdermal therapeutic system for administration of an active ingredient through the skin, comprising: (a) a back layer, (b) a reservoir present on the back layer, comprising the active ingredient, polyisobutylene, a gel former and a plasticizer, a portion of the active ingredient in the reservoir being present in the form of undissolved particles and the content of gel formers in the reservoir being at most 4% by weight (based in the total weight of the reservoir), the active ingredient being Fentanyl or an analogue thereof.

An object of the present application is a system for the transdermal administration of fentanyl or an analogue thereof for therapeutic purposes.

Fentanyl and its analogues, in particular alfentanil, carfentanil, lofentanil, remifentanil, sufentanil, trefentanil, and related compounds are potent synthetic opiates. Fentanyl and its analogues are highly efficacious and are rapidly metabolized. A problem with fentanyl is its relatively narrow therapeutic index. When the threshold values are exceeded undesired side effects occur, in particular impairment of respiration what can—unless suitable countermeasures are taken—cause death. The active ingredients are relatively expensive and there is a high risk of abuse. That's why fentanyl patches on the one hand have to ensure a very precisely controlled release of the active ingredient and on the other hand the product should be designed such that the active ingredient cannot be removed easily out of it for purposes of abuse.

Usually, the transdermal patch is a small adherent bandage containing the active ingredient to be delivered. These bandages can have various forms and sizes. The simplest type is an adhesive monolith comprising an active ingredient stock (reservoir) on a carrier. Usually, the reservoir is formed of the active ingredient in a pharmaceutically acceptable pressure-sensitive adhesive. However, it can also be formed of a non-adherent material the skin-contacting surface of which is provided with a thin layer of a suitable adhesive. The rate of administration of the active ingredient to the patient from these patches can vary in certain limits from person to person as well as from skin site to skin site depending on the permeability of the skin for the active ingredient.

More complex patches are multiple laminates or patches having an active ingredient stock (which can optionally be solved in a liquid) wherein a membrane controlling the release of the active ingredient can be arranged between the reservoir and the skin-contacting adhesive. This membrane is for the control and optionally reduction of the effects of variations of the skin permeability by lowering the rate of delivery in vitro and in vivo of the active ingredient from the patch.

The reservoir of the transdermal patches can contain the active ingredient either completely dissolved in the stock or it can contain an excess of undissolved active ingredient beyond its saturation concentration (depot patch). However, the presence of undissolved active ingredient or other constituents in a patch can cause stability and other problems in storage as well as in use. Also a difficulty is that it has to be ensured that sufficient active ingredient dissolves from the solid depot additionally to replace the delivered active ingredient. In the state of the art, active ingredient patches the reservoirs of which have solid active ingredient particles are often considered to be detrimental.

Various transdermal patches for the administration of fentanyl are known from the state of the art. WO 02/074286 describes a transdermal patch having a reservoir containing fentanyl wherein the reservoir has a polymeric composition, preferably polyacrylate in a uniform phase state being free of undissolved active ingredient. Here, a supersaturation should explicitly be avoided.

There are many experiments to prepare fentanyl patches also on the basis of matrix layer of polyisobutylene. First, such experiments are already described in the basic patent regarding fentanyl patches U.S. Pat. No. 4,588,580. This publication discloses a transdermal therapeutic system with a polyisobutylene matrix and mineral oil containing a 2% load of fentanyl. However, in the period following the development departed from polyisobutylene matrices and if any polyisobutylene matrices were used the attempt was made to completely solve the active ingredient in the polyisobutylene matrix.

A transdermal therapeutic system with a polyisobutylene matrix is described in Roy et al., Journal of Pharmaceutical Sciences, Vol. 85, No. 5, May 1996, pp 491 to 495. It is shown that with concentrations of fentanyl in the polyisobutylene matrix of more than 4% active ingredient is precipitating and this Roy et al. obviously considered negative. Roy et al. suggest matrix patches for the administration of fentanyl wherein the fentanyl is present completely dissolved.

In EP 1 625 854, US 2007/0009588, and US 2006/0013865 corresponding polyisobutylene matrices are suggested in case of which it is carefully to attend that the active ingredient is present completely dissolved in the polyisobutylene matrix. Occuring of crystals in the matrix is considered to be negative. The systems disclosed herein show a time-dependent releasing rate wherein the concentration of the fentanyl in the blood of a patient raises within the first 20 hours after administration; however, then does not remain constant as desired over a period of at least 2, better at least 3 days but drops during this period. Such patches are particularly not suitable to administer fentanyl over a longer period of e.g., 7 days.

DE 198 37 902 discloses transdermal therapeutic systems on the basis of polyisobutylene that are particularly suitable for the administration of clonidine; however, among the active ingredients mentioned also fentanyl is found. No examples of fentanyl patches are found in this publication; also an indication that the active ingredient should be present as solid in the disclosed patches is not found in this publication. The publication does not contain in vivo investigations regarding the release of the active ingredient from the patches. The polyisobutylene layer of these patches contains at least 5% by weight of a filler.

Despite all these experiments there is no patch until now on the basis of polyisobutylene that fulfills the requirements of the licensing authorities in view of the release of the active ingredient and the attainable plasma levels, respectively and hence would be marketable.

In view of the state of the art the object is to provide a transdermal therapeutic system for the administration of fentanyl or an analogue thereof through the skin formed on the basis of polyisobutylene and which does not show the problems of the state of the art. In particular, the patch should be able to release fentanyl or an analogue thereof, respectively with a lag time as short as possible and in particular steady over a long time, and not exhibit the time-dependent releasing rate that is found with polyisobutylene systems according to the state of the art. The plasma level should remain as constant as possible for as long as possible, but preferably over a period of about 30 hours after administration to about 70 hours after administration, but preferably also for more than 70 hours. In particular, the patch should be suitable to provide after administration analgesia for a period of time longer than 3 days, especially 4 to 7 days and more preferably for about 7 days.

Hence, the object of the present invention is a transdermal therapeutic system for the administration of an active ingredient through the skin comprising:
(a) a back layer;
(b) a reservoir on the back layer comprising the active ingredient, polyisobutylene, a gel former, and a plasticizer wherein a part of the active ingredient in the reservoir is present in the form of undissolved particles and the content of gel former in the reservoir is at most 4% by weight (based on the total weight of the reservoir),

wherein the active ingredient is fentanyl or an analogue thereof.

In addition to fentanyl according to the invention analogues of the fentanyl are preferred such as alfentanil, carfentanil, lofentanil, remifentanil, trefentanil or sufentanil wherein it is especially preferred that the active ingredient is fentanyl or sufentanil. In the following the invention is explained substantially with regard to the fentanyl. However, the embodiments apply correspondingly also to the analogues of fentanyl.

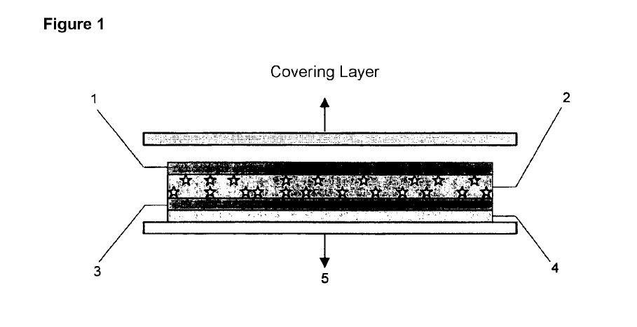

The construction of a preferred transdermal therapeutic system in cross-section is shown in FIG. 1. The transdermal therapeutic system is covered by means of a covering layer. The back layer (1) is located at the end of the patch that is in use opposite to the skin. The reservoir (2) is located at a side of the back layer (1) that faces in use the human skin. In a preferred embodiment the transdermal therapeutic system can have a membrane (3). The membrane (3) is arranged between the reservoir (2) and the skin to which the patch is to be applied. If a membrane (3) is present, preferably between the membrane (3) and the skin there is an adhesive layer (4) being free of active ingredient, alternatively but not preferred in this case an adhesive layer can also be applied next to the membrane and e.g., can surround it circularly.

In addition there is a stripping layer (5) on the side of the adhesive layer (4) opposite to the reservoir (2) that is peeled off before the use of the transdermal therapeutic system.

In a preferred embodiment in the transdermal therapeutic system there is provided a membrane (3) at the side of the reservoir (2) opposite to the back layer controlling the release of the active ingredient. It is the main purpose of the membrane to reduce the in vivo and in vitro releasing rate of the active ingredient from the patch. So, differences in the permeability for the active ingredient through the skin can be balanced. Preferably, the membrane is a microporous membrane.

Suitable membranes are known in the state of the art. In a preferred embodiment the membrane can contain or may be composed of polypropylene or polyethylene vinylacetate. An especially preferred material for the membrane is a microporous polypropylene film.

The thickness of the membrane is not particularly restricted and can e.g., be in the range of 10 μm to 100 μm, preferred less than 50 μm, e.g., about 25 μm. The pore size is preferably in the range of 0.001 to 0.025 μm², e.g., in the range of 0.002 to 0.011 μm², particularly about 0.005 μm². Also the shape of the pores is not particularly restricted, a rectangular shape is preferred.

Hence, a typical example of a suitable membrane is a microporous polypropylene film having a thickness of about 25 μm and a pore size of about 0.12 μm×0.04 μm, as marketed under the trade name Celgard 2400 from Celgard LLC, Charlotte, USA.

Optionally, the membrane can be pretreated according to known methods.

Between the membrane and the stripping film preferably there is located an active ingredient-free adhesive layer (4) allowing the adhesion of the patch on the skin. Alternatively, the adhesive layer can also be arranged around the membrane. Said adhesive layer can consist of various per se known pressure-sensitive adhesives wherein preferably polyisobutylene but also other substances as polybutene, various resins from mineral oil products, or suitable polyacrylates can be used. If a polyisobutylene is used for the adhesive layer as pressure-sensitive adhesive preferably the same polyisobutylene is used as was employed for the reservoir. The adhesive layer can contain the conventional additives for adhesive layers in active ingredient patches. The thickness of the adhesive layer (dry thickness) can vary in a range of about 10 μm to about 300 μm, preferably between about 70 μm and about 140 μm. When the amount of the adhesive layer is related to weight per area of the patch the amount of the adhesive layer is preferably about 10 mg/10 cm² to about 50 mg/10 cm², preferably about 20 to about 40 mg/10 cm² (ready-to-use i.e., dry patch).

On the adhesive layer or the self-adherent reservoir without membrane, respectively there is a stripping layer (release liner) identified with number 5 in FIG. 1. Preferably, said stripping layer is prepared of polymeric material that can optionally be metalized, too. Examples of preferably employed polymeric materials are polyurethanes, polyvinylacetate, polyvinylidene chloride, polypropylene, polycarbonate, polystyrene, polyethylene, polyethylene terephthalate, polybutylene terephthalate as well as paper optionally surface-coated with the corresponding polymers. Preferably, this is a stripping layer that is fluorinated or siliconized on one or both side(s). Especially preferred are commercially available fluorinated or siliconized polyester films such as the one-sided siliconized commercial products Primeliner 100 μm and Perlosic LF 75 μm (Loparex, NL and Perlen Converting AG, Switzerland).

The reservoir (3) comprises the active ingredient, preferably fentanyl, polyisobutylene, a gel former, and a plasticizer wherein the individual components are matched such that a part of the active ingredient (fentanyl) in the reservoir is present in the form of undissolved particles, so the content of fentanyl is above the saturation solubility of the fentanyl in the reservoir so that not all of the fentanyl is dissolved. This applies for room temperature, i.e. about 25° C., but preferably both room temperature and application temperature (skin temperature), i.e. about 34° C. (the skin temperature is somewhat below body temperature).

The plasticizer is a basically known compound employed in the state of the art in transdermal therapeutic systems as plasticizer. Preferably, the plasticizer is also designed to enhance the penetration of the active ingredient through the skin and in an especially preferred embodiment it regulates the solubility of the active ingredient in the reservoir such that a certain content of active ingredient is maintained in solution.

Preferably, the plasticizer is mineral oil, linseed oil, octyl palmitate, squalene, squalane, silicone oil, isobutyl myristate, isostearyl alcohol, and oleyl alcohol wherein mineral oils are the preferred plasticizers. This oils which are also referred to as thin paraffins are colorless clear hydrocarbons. They are obtained from the petroleum distillation fractions boiling above 300° C. and are liberated from solid hydrocarbons by cooling. They are refined by extraction with solvents as well as by treatment with bleaching earths and/or sulfuric acid. Suitable mineral oils are both chemically and biologically stable and prevent bacterial growth. By suitable fractionation mineral oils can be obtained that are liquid around body temperature, i.e. at about 35 to 37° C. and are solid at lower temperatures, especially at temperatures below 20° C. Preferred is to choose a mineral oil having a liquefaction point of about 30-35° C.

Preferably, the plasticizer is present in the reservoir in an amount ranging from 10 to 60% by weight, more preferably from 25 to 50% by weight, in particular ranging from 30 to 40% by weight (based on the total weight of the reservoir).

In the transdermal therapeutic system the reservoir has to contain an amount of fentanyl or an analogue thereof sufficient to induce analgesia in a human being and to maintain it for at least two days, preferably at least three days, more preferably at least 4 days, and in particular about 7 days (based on the point of administration of the patch). Preferably, the reservoir contains an amount of fentanyl or an analogue thereof sufficient to induce analgesia and to maintain it for a period of at least three days, in particular three to seven days, especially preferred about 7 days.

Since a part of the active ingredient is present in the patch according to the invention in undissolved form the patch is particularly suitable for a very long employment even for more than 3 days, for example up to 7 days. Namely, the content of active ingredient can readily be increased being not possible in case of patches containing the active ingredient in completely dissolved form. When increasing the fentanyl content above the saturation solubility in such patches it has to be expected that the active ingredient crystallizes out having negative effects on releasing in such systems.

The absolute amount of active ingredient to be employed depends on various factors, in particular the size of the patch to be used and the duration of use. Preferably, the transdermal therapeutic system contains the active ingredient in an amount of 5 to 30% by weight, preferably of 5 to 20% by weight, more preferably in an amount of 5 to 15% by weight, based on the total weight of the reservoir. Preferably, there result weights per unit area ranging from 20 to 100 g/m², more preferably 25 to 80 g/m², in particular in the range of 30 to 70 g/m². Preferably, the reservoir has a thickness (dry thickness) in the range of 20 to 400 μm, more preferred in the range of 30 to 200 μm, in particular in the range of 40 to 100 μm.

The reservoir of the patch according to the invention also contains a gel former. Preferably, this is a gel former with a particulate structure having on its surface a high concentration of polar groups. Said groups cause correspondingly high interfacial tensions towards the oils which are partially compensated by agglomeration of the particles among themselves to gel skeletons. Accordingly, the greater the difference in polarity between the oils and the skeleton former surface the stronger are the gel skeletons. According to the invention it is preferred to employ as the gel former highly disperse silica or colloidal silica. The particle size is preferably in the nano-area and is e.g., in the range of 400 to 1500 nm, in particular in the range of 500 to 1000 nm. For example, colloidal silica is marketed under the designation Cab-O-Sil® and is a known thickener for mineral oil. Another example for a suitable gel former is bentonite. Also sodium carbomer known as gel former can be used. Preferably, the gel former is used in an amount of 0.1 to 4.0, more preferably 0.5 to 2.0% by weight, based on the total weight of the reservoirs.

In a preferred embodiment the polyisobutylene of the reservoir consists of two different types of polyisobutylene with different molecular weights. This means, that one type of the polyisobutylene has a peak in the distribution of the molecular weight that differs from the peak of the molecular weight of the other type of the polyisobutylene. So, the two polyisobutylenes have a different mean molecular weight (weight average mean molecular weight M_{w}). Preferably, the polyisobutylene (high-molecular polyisobutylene) has a mean molecular weight of 150,000 to 10,000,000, especially preferred of 500,000 to 10,000,000, and the second polyisobutylene has a lower mean molecular weight (low-molecular polyisobutylene) in the range of 15,000 to 100,000, preferably of 20,000 to 80,000. As a rule, the weight average mean molecular weight M_{w} is determined via GPC. In particular, the polyisobutylene with the lower mean molecular weight is responsible for the stickiness of the patch. The two polyisobutylenes with different molecular weight can be mixed with each other wherein the ratio of the polyisobutylene having the higher molecular weight to the polyisobutylene having the lower molecular weight is preferably in the range of 0.05:1 to 20:1, especially preferred of 0.5:1 to 2:1, in particular about 1:1, however mixtures of polyisobutylenes having different molecular weights are also commercially available. A particularly suitable commercial product is the product DuroTak®87-616A from National Starch.

In a preferred embodiment also the adhesive layer is a layer of polyisobutylene wherein preferably a mixture of two polyisobutylenes having different molecular weights is employed as in the reservoir. However, the ratio of the two polyisobutylenes having different molecular weights in the adhesive layer preferably differs from that in the reservoir layer. It is particularly preferred that in the adhesive layer there are employed the same polyisobutylenes as in the reservoir but in a different ratio. So, in the adhesive layer the ratio of the polyisobutylene having the lower molecular weight to the polyisobutylene having the higher molecular weight is preferably in the range of 20:7 to 2:1, preferably 15:1 to 5:1, in particular about 10:1 or 9:1.

Moreover, the adhesive layer can contain further conventional excipients and additives. When the transdermal therapeutic system has a membrane then in particular the adhesive layer can also contain plasticizers and/or gel formers.

Based on the total weight of the reservoir the portion of the polyisobutylene, preferably of both polyisobutylenes having different molecular weights, is preferably 30 to 80% by weight, e.g., 40 to 65% by weight, preferably 50 to 60% by weight, based on the total weight of the reservoir.

As long as in the context of this description the term “total weight of the reservoir” or an amount referring to the reservoir, etc. is used this means the dry weight, i.e. the weight of the reservoir in the ready-to-use patch, unless otherwise disclosed or apparent.

It is substantial for transdermal therapeutic systems according to the invention that the reservoir contains sufficient active ingredient so that this contains active ingredient particles.

In the production of the patch according to the invention the active ingredient is preferably used in a micronized form having a mean particle size of 50 μm or less, preferably having a mean particle size of 20 μm or less. Also, in the reservoir layer of the patch (the matrix layer) the active ingredient is present in a micronized form, however there can result minor deviations of the particle size by rearrangement reactions when storing the patch. However, also, within the patch the mean particle size of the active ingredient particles is preferably less than 100 μm, more preferred less than 50 μm, and in particular about 20 μm or less. For the production of the patch according to the invention there is preferably used micronized fentanyl having a mean particle size of 1 μm or more, more preferred 2 μm or more. Also, said mean particle sizes are preferably found in the finally produced patches. In the reservoir layer of the transdermal therapeutic system the particle size and the particle-size distribution of the active ingredient particles can be determined by conventional light microscopy. The evaluation is carried out with conventional computer programs (image processing systems) that as a rule are adapted to the microscopes used. Unless otherwise indicated or apparent the particle size refers to the particle diameter.

As the starting material for the micronized fentanyl there is used the commercially available fentanyl which is per se suitable for clinical application. Typically, such fentanyl shows a distribution of the particle size such that 100% of the particles are smaller than 675 μm. About 90% of the particles are smaller than about 90 μm, and 50% of the particles are smaller than about 25 μm.

According to the invention any known micronization process providing the desired particle size can be used. It is preferred to use fentanyl that was micronized by means of a conventional “jet mill”, e.g., a jet mill of the AS type by Hosokawa Alpine AG.

By the micronization process used in accordance to the invention the size of the fentanyl particles is preferably changed such that the mean particle size is in the above-mentioned ranges. It is also preferred that 100% of the particles are smaller than 50 μm, in particular smaller than 20 μm. Preferably, about 90% of the micronized particles are smaller than 12 μm, and about 50% of the particles are smaller than 6 μm.

There are various methods for determining the particle size and the particle-size distribution of the active ingredient, for example the light scattering method as used in the devices of Malvern Instruments, e.g., the “Malvern MasterSizer X”, the mechanical sieve shaking method as used by FMC for determining the particle-size distribution of its AVICEL PH® products, or also “air jet” sieve analyses which can be performed with an ALPINA® “air jet” model 200. Preferably, the determination of the particle sizes and distributions is carried out microscopically by light microscopy and a suitable image processing software.

Unless indicated otherwise the (mean) particle sizes and particle-size distributions, respectively are determined with an optical microscope, a camera attached thereto, and an automated evaluation software. Here, the intended test conditions that as a rule are predetermined by the manufacturers of the microscopes have to be fulfilled.

If the active ingredient is defined by the indication of the mean particle size and the particle-size distribution the micronized active ingredient used according to the invention has preferably a mean particle size of 20 μm or less and it is preferred that the active ingredient has a grain-size distribution (particle-size distribution) wherein less than 10% of the particles have a size of 25 μm or more and less than 10% of the particles have a size of 1 μm or below. Further preferred is an active ingredient having a mean particle size of 15 μm. Preferably, such an active ingredient has a grain-size distribution wherein less than 2% of the particles have a size of 20 μm or more and less than 50% of the particles have a size of 5 μm or below. The grain-size distribution for said active ingredient should be as narrow as possible.

On the side of the reservoir that is in use turned away from the human skin there is a back layer being in preferred embodiment occlusive, i.e. ending. In a preferred embodiment such back layers can consist of polyolefins, in particular polyethylene, or of polyester as well as polyurethanes. Also, layers containing several different polymers arranged one upon the other may be employed advantageously. A particularly preferred material for the back layer is a polyolefin marketed by Mylan Technologies Inc. under the designation Mediflex®1000. Other suitable materials comprise cellophane, cellulose acetate, ethyl cellulose, plasticizer-containing vinyl acetate vinyl chloride copolymers, ethylene vinyl acetate copolymers, polyethylene terephthalate, nylon, polyethylene, polypropylene, polyvinylidene chloride, ethylene methacrylate copolymer, paper which optionally can be coated, textile fabrics, aluminum film, and polymer metal composite materials. Especially preferred are polyester films such as polyethylene therephthalate films. The thickness of the back layer is, e.g., 10 μm to 50 μm, e.g., about 20 μm nominal thickness as common in the state of the art.

On the back layer of the patch there is preferably a covering layer that should in particular prevent that the patch adheres to the package in case small amounts of polyisobutylene or adhesive pass out. Preferably, the covering layer lies loose on the back layer and is kept by electrostatic forces. Such covering layers are known in the state of the art, e.g., from EP 1 097 090, which is incorporated in its entirety herein by reference. The covering layer is non-stick, e.g., siliconized or fluorinated at least on the side lying on the back layer.

The production of a preferred transdermal therapeutic system is carried out by dispersing the components for the reservoir, i.e., fentanyl and the gel former, in an organic medium as heptane and mixing the mineral oil and polyisobutylene in an organic medium, preferably the same as before. Then, fentanyl and the gel former are dispersed in the mixture of polyisobutylene and mineral oil. In the production of the reservoir preferably a volatile organic medium such as for example heptane is employed. Then, said mixture is applied as uniform layer onto the back layer and dried. If it is desired to apply a membrane this is applied on the side of the reservoir opposite to the back layer.

In a separate operation the (active ingredient free) adhesive layer is optionally applied to the stripping film and is allowed to dry.

Subsequently, the components obtained in both processing steps are laminated with each other in a way that the adhesive layer is applied to membrane, if such a membrane is provided. In the embodiments wherein no membrane is employed the adhesive layer is laminated directly to the reservoir (if in this case an adhesive layer is present). Thereafter, pieces of the desired size can be die-cut from the final laminated film and packaged.

In the individual processing steps the organic solvents required to dissolve the polyisobutylene and to disperse the other constituents are removed by subjecting the products raising temperatures, optionally also using a low pressure.

With the transdermal therapeutic system according to the invention the plasma levels can be adjusted very good. Hence, according to the invention there are preferred transdermal therapeutic systems as described and claimed in this application providing a steady-state plasma level in the range of 200 pg/ml to 500 pg/ml, preferred of 250 pg/ml to 350 pg/ml (pg=pico grams) when administered to a patient. Particularly preferred are those transdermal therapeutic systems as described in this application and claimed in the claims wherein the following applies:

When a regression line is drawn through the plasma level values in the range of 30 hours to 70 hours (at least one measuring point every 5 hours) the plasma level value on the regression line at 30 hours deviates from the plasma level value on the regression line at 70 hours by not more than 10%.

That shows the very high consistency of the plasma level values of the fentanyl obtainable with the formulations according to the invention.

The following examples explain the invention but are not restricting.

### EXAMPLE 1

For the production of a transdermal patch at first fentanyl having a mean particle size of 20 μm (Gesellschaft für Mikronisierung mbH, jet mill, AS, Hosokawa Alpine AG) was dispersed in heptane with the polyisobutylene (DuroTak®87-616A), silica (Cab-O-Sil M-5P) as the gel former, and mineral oil “Klearol” as the plasticizer. The mixture was applied as a thin layer to the back layer and was allowed to dry so that a weight per unit area of about 55 g/m² resulted. Thereafter, a microporous polypropylene film (Celgard 2400) was applied as membrane.

In parallel, only polyisobutylene (DuroTak®87-616A, dissolved in heptane) was applied as a thin layer to the stripping layer and was allowed to dry so that a weight per unit area of about 30 g/m² resulted. The tolerance of the weights per unit area was 10%.

After drying both layers they were laminated with each other whereas the membrane was connected with the reservoir using suitable pressures.

Subsequently, rectangular patches having rounded edges with a size of 10 cm² were die-cut and packaged with the covering film. The final product had the following composition:

| | | |
|---|---|---|
| | Nominal | Concentration (%) of the |
| Name of the | release 25 μg/h | ready-to-use (dry) |
| constituent | P -atch size 10 cm² | reservoir |

Fentanyl-containing reservoir

| | | |
|---|---|---|
| Fentanyl | 4.95 mg | 9% |
| DuroTak ® 87-616A | 30.0 mg | 54.5% |
| Cab-O-Sil M-5P | 0.55 mg | 1% |
| Klearol (mineral oil) | 19.5 mg | 35.5% |
| Heptane* | — | — |

Adhesive layer

| | | |
|---|---|---|
| DuroTak ® 87-616A | 30.0 mg | 100% |
| Heptane* | — | — |

Layer materials

| | | |
|---|---|---|
| Celgard 2400 | 10.0 | cm² |
| Hostaphan MN 19 MED | 10.0 | cm² |
| Primeliner 100 μm PET | 15 | cm² |
| C1S | | |
| PETP film, 36 μm, | 15 | cm² |
| transparent | | |
| Aluminum package, | 1 | bag |
| white | | |

*not present in the final product

The components employed in the example can be explained in more detail as follows:

| Component | | |
|---|---|---|
| designation | Chemical description | Function |
| DuroTak ® 87- | polyisobutylene adhesive | pressure-sensitive |
| 616A | dissolved in n-heptane | adhesive |
| Cab-O-Sil M-5P | colloidal silica | gel former |
| Klearol | light mineral oil | plasticizer |
| Celgard 2400 | microporous polypropylene | membrane |
| | film | |
| Hostaphan MN 19 | polyethylene terephthalate film | back layer |
| MED | having a nominal thickness of | |
| | 19 μm | |
| Primeliner 100 μm | polyester film having a nominal | stripping layer |
| PET C1S | thickness of 100 μm, one side | |
| | siliconized | |
| PETP film, 36 μm, | polyester film having a nominal | covering layer |
| transparent | thickness of 36 μm, one side | |
| | siliconized | |
| Aluminum package, | package of hot-sealing | packaging |
| white | adhesive | material |

### EXAMPLE 2

Transdermal patches prepared in accordance to the procedure described in example 1 were investigated in a pilot study.

In the pilot study six healthy voluntary subjects were employed. There were tested two different patches in total. The control was a state of the art product with the designation Durogesic SMAT 25 μg/h, a monolithic transdermal system having a polyacrylate matrix. Said patch was compared with the patch according to the invention as described in example 1. Here, the nominal release is 25 μg fentanyl per hour.

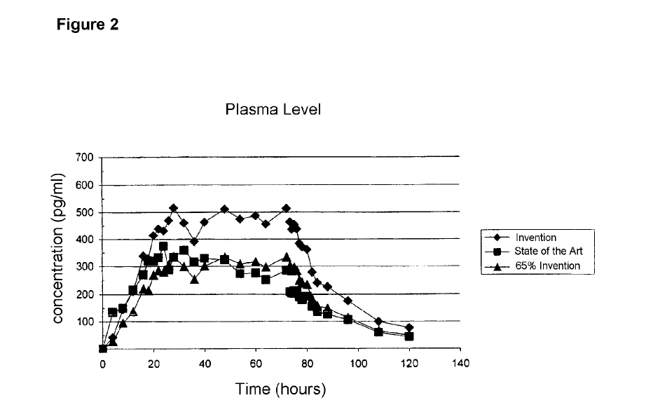

The patches were adhered to the subjects and subsequently the concentrations of fentanyl in the blood were measured. The means of six measures each were plotted in the release curve in accordance to FIG. 2.

The release curve in accordance of FIG. 2 shows that the patch according to the invention (—♦— invention) exhibits a clearly higher concentration of fentanyl in the plasma of the subjects and that the concentration is maintained constant even better over about three days as with the polyacrylate patch that got an approval for drugs as commercial product despite the not completely constant release.

Hence, a patch having only 65% of the size of the patch according to the invention exhibits a plasma level in the subjects corresponding to the value of the state of the art in connection with the better constancy as with the polyacrylate patch. These values are also recorded in FIG. 2.

This example clearly shows that the transdermal patch according to the invention has considerably improved release in comparison to a state of the art product. Hence, smaller patches can have the same efficacy like the greater patches known from the state of the art. In addition, the blood level curve shows that the active ingredient is used more efficiently.

Moreover, it was surprisingly shown that in the patches according to the invention the effect described for the polyisobutylene patches of the state of the art, namely a time dependency of the release within the first 3 days after administration is rather not shown but the plasma concentration remains constant over at least 3 days without showing a slow dropping of the plasma concentration after an initial rapid increase over the first 20 hours. This is surprising and exceptionally advantageous in the polyisobutylene patches according to the invention which are hence superior even to the polyacrylate based fentanyl patches present on the market at present.

1. A transdermal therapeutic system for the administration of an active ingredient through the skin comprising:
(a) a back layer; and
(b) a reservoir disposed on said back layer and comprised of: (i) an active ingredient comprising fentanyl or an analogue thereof, (ii) polyisobutylene, (iii) a gel former, and (iv) a plasticizer, wherein some of the active ingredient in the reservoir is present in the form of undissolved particles and the gel former in the reservoir constitutes at most 4% by weight based on the total weight of the reservoir.

2. The transdermal therapeutic system according to claim 1, characterized in that the active ingredient is fentanyl or sufentanil.

3. The transdermal therapeutic system according claim 1, wherein the side of the reservoir opposite to the back layer is provided with a microporous membrane.

4. The transdermal therapeutic system according to claim 1, wherein said reservoir is provided with a stripping layer that acts as a release liner.

5. The transdermal therapeutic system according to claim 4, characterized in that the reservoir contains an amount of fentanyl or of an analogue thereof sufficient to induce analgesia and to maintain it for a period of three to seven days.

6. The transdermal therapeutic system according to claim 1, characterized in that the plasticizer is mineral oil.

7. The transdermal therapeutic system according to claim 1, characterized in that the gel former is colloidal silica or bentonite.

8. The transdermal therapeutic system according to claim 1, characterized in that the undissolved active ingredient particles in the reservoir are present in a micronized form having a mean particle size of 50 mm or less.

9. The transdermal therapeutic system according to claim 1, characterized in that the polyisobutylene is a mixture of two polyisobutylene polymers having different molecular weights.

10. The transdermal therapeutic system according to claim 1, characterized in that the side of the back layer opposite to the reservoir is provided with a covering layer.

11. The transdermal therapeutic system according to claim 1, characterized in that the back layer is an occlusive back layer.

12. The transdermal therapeutic system according to claim 1, characterized in that the active ingredient in the reservoir is present in a concentration ranging from 3% by weight to 20% by weight, based on the total weight of the reservoir.

13. The transdermal therapeutic system according to claim 1, characterized in that the plasticizer is present in the reservoir in an amount ranging from 10 to 60%, based on the total weight of the reservoir.

14. The transdermal therapeutic system according to claim 1, characterized in that the polyisobutylene is present in the reservoir in an amount ranging from 30 to 80%, based on the total weight of the reservoir.

15. The transdermal therapeutic system according to claim 1, wherein said system further comprises an adhesive layer that is free of undissolved active ingredient.

16. The transdermal therapeutic system according to claim 15, characterized in that the adhesive layer is polyacrylate or a polyisobutylene.

17. The transdermal therapeutic system according to claim 15, characterized in that the area of the adhesive layer corresponds to the area of the reservoir.

18. The transdermal therapeutic system according to claim 8, wherein the undissolved active ingredient particles in the reservoir present in a micronized form have a mean particle size of 20 mm or less.
